(19) United States
(12) Patent Application Publication
(10) Pub. No.: US 2005/0244368 A1
(43) Pub. Date: Nov. 3, 2005

(54) Pre-endoscopic use of polyethylene glycol compositions

(76) Inventor: Dinesh S. Pashankar, Iowa City, IA (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/836,131

(22) Filed: Apr. 29, 2004

(51) Int. Cl. A61K 31/765

(52) U.S. Cl. 424/78.38

(57) ABSTRACT

A method to induce bowel cleansing which employs electrolyte-free polyethylene glycol containing compositions is provided.

### BACKGROUND OF THE INVENTION

Colonoscopy is an established procedure for investigating large bowel and terminal ileal diseases in infants and children. Successful visualization of the mucosa requires adequate bowel preparation to clean the colon. There are many bowel preparations available for use in children (Sondeimer et al., 1991; Barrish et al., 1993; Dahshan et al., 1999; Gremse et al., 1996; Silva et al., 1997; Pinefield et al., 1999). One standard preparation involves consumption of a large volume of polyethylene glycol-electrolyte lavage solution (PEG-ELS) over a few hours (Sondeimer et al., 1991). Another method of bowel cleansing uses several doses of laxatives such as magnesium citrate or senna along with a clear liquid diet for 2 to 3 days, and multiple enemas prior to the colonoscopy (Barrish et al., 1993; Dahshan et al., 1996). Oral sodium phosphate solution has also been used successfully in children for colonoscopy (Gremse et al., 1996; Silva et al., 1997). Despite the availability of various bowel preparations, none of them is completely safe, effective and acceptable to children. In a recent study comparing different bowel preparations in children, it was concluded that no current bowel preparation met these criteria (Dahshan et al., 1999).

Polyethylene glycol 3350 (PEG) is a relatively new osmotic laxative that has been successfully used to treat constipation and encopresis in children (Pashankar et al., 2001; Loening-Baucke, 2002; Gremse et al., 2002). PEG is a component of PEG-ELS (with electrolytes) that has long been used as a bowel preparation for colonoscopy in children (Sondeimer et al., 1991). PEG has been shown to be an effective and safe laxative and is well accepted by children due to its palatability (Pashankar et al., 2001; Loening-Baucke et al., 2002; Gremse et al., 2002; Pashankar et al., 2003).

What is needed is an improved bowel cleansing protocol, e.g., one that is safe and effective for children.

### SUMMARY OF THE INVENTION

The invention provides a method to induce bowel cleansing. The method includes administering to a patient in need thereof, e.g., prior to endoscopy, a composition comprising an effective amount of electrolyte-free PEG. Indications for endoscopy include, but are not limited to, diarrhea, abdominal pain, blood in stool, surveillance for polyps, e.g., due to a family history, weight loss, and/or cancer surveillance, e.g., in adults age 50 or older. In one embodiment, the amount is effective to reduce or eliminate stool in the colon, e.g., cecum and/or terminal ileum, of the patient at the time of endoscopy. For example, the amount is effective to eliminate stool or yield only a small amount of thin fecal fluid in the bowel of the patient. In one embodiment, the patient is a child, e.g., from about 1 to 20 years of age. In another embodiment, the patient is an adult, i.e., over 20 years old. In one embodiment, electrolyte-free PEG is administered in 2 to 3 divided doses per day. The effective amount may be administered over 1 or more days, for instance, administered over 2 or more days, e.g., administered over 4 or more days. Generally, larger dosages are needed for shorter periods of time. For example, if about 100 g/day of electrolyte-free PEG is administered for 4 days to cleanse the bowel, then larger dosages are needed to achieve the same effect in less than 4 days, e.g., about 150 to about 200 g/day. When electrolyte-free PEG is administered for more than 1 day, a patient may enjoy a normal diet, i.e., one which includes solids, up until the day before the endoscopic procedure.

As described herein, to assess safety, efficacy and acceptance of PEG 3350 without electrolytes for bowel preparation for colonoscopy in children, in a prospective study, 46 children (mean age 11.2 years; range 2.8 to 17.8) were given PEG at a dose of 1.5 g/kg/day for 4 days prior to colonoscopy. Patients were allowed to mix PEG without electrolytes in the beverage of their choice. Stool frequency and adverse effects were monitored during PEG therapy. Compliance, tolerance and quality of colonic preparation were assessed. Moreover, serum electrolytes were measured before and after PEG therapy in 29 children. It was found that daily stool frequency increased with PEG therapy from baseline of 2.6±0.3 to 3.0±0.5 on day 1, 4.6±0.4* on day 2, 5.5±0.7* on day 3, and 6.0±0.6* on day 4 (*=P<0.001 for difference vs. baseline). The colonic preparations were rated as excellent or good in 91% and 95% in the right and left colon, respectively, at endoscopy. Adverse effects were mild nausea (13%), abdominal pain (11%), and vomiting (11%). Electrolyte profile revealed small, clinically insignificant changes with PEG therapy. Compliance and tolerance were rated as excellent by 89 and 85% of patients, respectively. Thus, electrolyte-free PEG 3350 can be used as an effective and safe bowel preparation that is well accepted by children for colonoscopy.

The invention also provides a method of bowel preparation prior to colonoscopy. The method includes administering to a patient, e.g., a child, for two or more days prior to colonoscopy a composition comprising electrolyte-free PEG effective to prepare the bowel for colonoscopy. In one embodiment, the electrolyte-free PEG is administered in 2 to 3 divided doses per day. In one embodiment, the effective amount is administered over 2 or more days, e.g., over 4 days. When electrolyte-free PEG is administered for more than 1 day, a patient may enjoy a normal diet, i.e., one which includes solids, up until the day before the colonoscopic procedure.

Also provided is a method for intestinal lavage. The method includes administering to a patient in need thereof a composition comprising an effective amount of electrolyte-free PEG. In one embodiment, the patient is a child. In another embodiment, the patient is an adult. In one embodiment, electrolyte-free PEG is administered in 2 to 3 divided doses per day. The effective amount may be administered over 1 or more days, for instance, administered over 2 or more days, e.g., administered over 4 or more days.

Further provided is a method in which a patient is identified as a candidate for colonoscopy. Indications for endoscopy include, but are not limited to, diarrhea, abdominal pain, blood in stool, surveillance for polyps, weight loss, and/or cancer surveillance. Identified patients ingest an amount of electrolyte-free PEG effective to cleanse the bowel, e.g., an amount effective to eliminate stool or yield only a small amount of thin fecal fluid in the bowel of the patient at the time of endoscopy. The effective amount may be administered over 1 or more days and preferably over 2 or more days.

Also provided are prepackaged daily doses of electrolyte-free PEG, e.g., having from about 5 g to about 200 g, or any integer in between, of electrolyte-free PEG per dose. For instance, the prepackaged dose may be an individual daily dose or a series of daily doses, e.g., for 2 or more days. The prepackaged daily doses may be packaged with one or more beverages. In one embodiment, the prepackaged doses are in tablet form.

### BRIEF DESCRIPTION OF THE DRAWINGS

FIG. 1. Increase in daily stool frequency (mean±SEM) with PEG therapy over 4 days of bowel preparation compared to baseline. *=difference from baseline value.

### DETAILED DESCRIPTION OF THE INVENTION

The invention provides compositions, methods and protocols to achieve bowel cleansing so that optimally little or no time or effort is employed to clean the bowel during endoscopy, e.g., no stool remains in the colon, e.g., cecum or terminal ileum, only a small amount of thin fecal fluid remains in the colon, e.g., cecum or terminal ileum, or residual liquid fecal matter is readily removed by suction. The compositions include PEG and the PEG compositions of the present invention are substantially free of electrolytes; by “substantially free” is meant containing less than 5%, e.g., less than about 4%, 3%, 2% or 1%, by weight, and desirably as close to 0% as practicable. The PEG compositions of the present invention are preferably salt-free, as salts may cross the intestinal mucosa and, due to solvent drag, withdraw water from the intestinal contents, which can increase or induce constipation.

Any food- or pharmaceutical-grade PEG polymer may be employed in the compositions contemplated herein. Polymers of relatively high molecular weight (e.g., above about 900) that are solid at room temperature (i.e., about 25° C.) and soluble in (or miscible with) an aqueous medium at room temperature are preferred. Polymers having an average molecular weight of at least 1000 (and generally no greater than 20,000) are exemplary, while an average molecular weight between about 3000 and 8000 is preferred. In one embodiment, PEG 3350 (the numeric designation identifying the average molecular weight) is employed. Moreover, mixtures of PEGs of different molecular weight may be employed. Further, different molecular weight PEGs may be administered consecutively, e.g., on day 1 PEG having an average molecular weight of at least 1000 is administered while on subsequent days, PEG having an average molecular weight of at least 3000 is administered. Alternatively, on day 1 PEG having an average molecular weight of at least 3000 is administered while on subsequent days, PEG having an average molecular weight of at least 1000 is administered. Moreover, different combinations of PEG-ELS and electrolyte-free PEG may be employed, e.g., as a mixture or consecutively.

Compositions suitable for use in the present invention include those wherein PEG is administered in an effective amount to achieve its intended purpose. More specifically, an “effective amount” includes an amount effective to clean the bowel of a mammal prior to endoscopy. Determination of an effective amount is well within the capacity of persons skilled in the art, especially in light of the detailed disclosure provided herein.

The pharmaceutically active compounds of the invention can be formulated as pharmaceutical compositions and administered to a mammalian host, such as a human patient, in a variety of forms adapted to the chosen route of administration, e.g., orally. For oral therapeutic administration, the active compound may be combined with one or more excipients or diluents and used in the form of ingestible tablets, buccal tablets, troches, capsules, elixirs, suspensions, syrups, wafers, and the like. Thus, the present compositions may be systemically administered, e.g., orally, for instance, via a feeding tube, indwelling catheter, in a beverage or a tablet, optionally in combination with a pharmaceutically acceptable vehicle such as an inert diluent or an assimilable edible carrier. When the pharmaceutically active agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 100% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus or paste.

The pharmaceutically active agents may be enclosed in hard or soft shell gelatin capsules, may be compressed into tablets, incorporated into an aqueous medium, e.g., a beverage, or may, up until the day prior to endoscopy, be incorporated directly with the food of the patient's diet. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil. Various other materials may be present as coatings or to otherwise modify the physical form of the solid unit dosage form. For instance, tablets, pills, or capsules may be coated with gelatin, wax, shellac or sugar and the like. Moreover, enteric coated caplets or tablets of an agent of the invention are designed to resist disintegration in the stomach and dissolve in the more neutral to alkaline environment of the duodenum. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations. Under ordinary conditions of storage and use, these preparations may contain a preservative to prevent the growth of microorganisms.

One or more suitable unit dosage forms comprising the pharmaceutically active agents of the invention may optionally be formulated for sustained release The formulations can be so constituted that they release the active ingredient only or preferably in a particular part of the intestinal tract, possibly over a period of time. The coatings, envelopes, and protective matrices may be made from polymeric substances, for example, natural gels, synthetic polymer gels or mixtures thereof, and including polylactide-glycolates, liposomes, microemulsions, microparticles, nanoparticles, or waxes. Thus, sustained release dosage forms of the invention may comprise microparticles and/or nanoparticles having a pharmaceutically active agent dispersed therein. The dosage forms of this aspect of the present invention may be of any configuration suitable for sustained release. Preferred sustained release dosage forms have a biodegradable structure designed to biodegrade over a period of time preferably between from about 0.25 to about 10 days, preferably from about 0.5 to about 5 days, or from 1-3 days; or non-biodegradable structure to allow agent diffusion to occur over a time period of between from about 0.25 to about 10 days, preferably from about 0.5 to about 5 days, which dosage form is biocompatible with the local physiological environment into which the dosage form to be administered, optionally including yielding biocompatible biodegradation products.

Useful dosages of the pharmaceutically active compounds of the invention can be determined by comparing their in vivo activity in animal, e.g., human, models. The amount of the compound required for use may vary with the route of administration, and the age and condition of the patient, and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Compositions according to the present invention may be prepared by dispersing and/or dissolving the PEG in water or other aqueous medium to formulate a relatively smooth, palatable drink. PEG is an osmotically active agent that is not significantly absorbed in the intestine, and may therefore be taken in dosages ranging from about 5 to about 200 g up to four times per day. “About” as used herein with respect to amounts includes amounts that are within 1% to 10%, e.g., within 5%, of the stated amounts. Preferably, anywhere from 10 to 30 g of PEG in solid form are conveniently dispersed/dissolved in from about 6 to about 10 fluid oz. (i.e., about 10-12 times the weight of the solid PEG) of an aqueous medium. In one embodiment, the mixture is ingested orally from one and up to four or more times per day. PEG may be furnished in solid form for dispersal in a suitable liquid (e.g., water or juice), or in pre-mixed liquid form, or in solid form for oral ingestion (e.g., as solid wafers, capsules, or tablets).

In general, a suitable dose will be in the range of from about 0.1 to about 5 g/kg, e.g., from about 0.5 to about 2.5 g/kg of body weight per day, such as 1.0 to about 2.0 g per kilogram body weight of the recipient per day, preferably in the range of 1.25 to 1.75 g/kg/day, most preferably in the range of 1.3 to 1.6 g/kg/day. Generally, the dosage per day is about 1 to 250 g/day, e.g., about 5 to about 200 g/day, such as about 50 to about 150 g/day, including about 75 g to about 125 g/day. In one embodiment, where the PEG is dispersed in an aqueous medium, the dosage of PEG is administered in a volume of about 10 mL/day to about 2000 mL/day, e.g., 100 mL/day to about 1500 mL/day.

The invention will be further described by the following non-limiting example.

### EXAMPLE

Methods

Patients

Children (age 2 to 19 years) undergoing colonoscopy were eligible for the study. Patients were excluded if they had known allergy to polyethylene glycol, metabolic or renal disease, or if they required emergency colonoscopy. Forty-six patients were enrolled in the study over 8 months. The main indications for colonoscopy included diarrhea (33%), abdominal pain (30%), and blood in stools (26%). Colonoscopies were performed by 1 of 3 physicians under conscious sedation with midazolam and meperidine.

The study was approved by the institutional review board of the University of Iowa College of Medicine. Informed consent was obtained from parents of patients and assent was obtained from children older than seven years of age.

Bowel Preparation Regimen

Patients were started on PEG without electrolytes (supplied in the form of a virtually tasteless powder, i.e., MiraLax®, from Braintree Laboratories, Braintree, Mass.; “PEG” for the remainder of the Example) 1.5 g/kg/day up to a maximum of 102 grams per day for 4 days prior to colonoscopy. Patients and their parents were instructed to dissolve 17 grams (one capful) of PEG powder in 240 ml of water or other beverage according to manufacturer's directions and to give the appropriate amount of PEG solution in 2 or 3 divided doses. Families were allowed a free, choice of beverage to suit each child's preference and were encouraged to use different beverages during the bowel preparation. Patients were allowed a normal diet except for clear fluids on day 4 (one day prior to colonoscopy). All patients and parents received a detailed explanation of the bowel preparation and written instructions were also provided. Patients and parents were given a diary to record the number of daily stools and stool consistency over the 4 days of bowel preparation. Stool consistency was assessed on a 1 to 5 scale as follows: 1- hard, 2-firm, 3-soft, 4-loose, and 5-watery. On the day of colonoscopy, PEG was not given and patients were allowed to have clear fluids up to 6 hours before the procedure. Normal saline enemas were given prior to colonoscopy in the first 25 patients as a part of the protocol.

Assessment

Baseline blood tests were performed to measure serum creatinine, blood urea nitrogen, serum osmolality and serum electrolytes before starting the bowel preparation. In 17 patients, baseline blood tests were not performed because they were enrolled in outreach clinics or they refused to have blood tests. On the day of colonoscopy, stool diaries were collected and all patients were given a questionnaire. Data were collected about the daily PEG dose, beverages used to prepare the PEG solution, and the palatability of the PEG solution. The baseline daily stool frequency and consistency over the last month prior to PEG therapy was noted. Patient compliance and tolerance to PEG were assessed by self-report as excellent, good, fair or poor. Ease of bowel preparation was rated as easy, okay, unpleasant or difficult. Parents were asked about any possible adverse effects seen over 4 days of PEG therapy on a scale of mild, moderate, or severe. Specific note was made of nausea, vomiting, abdominal pain, or bloating associated with PEG over 4 days. Patients were asked about willingness to retake the PEG bowel preparation for future colonoscopy if needed and about their preference of bowel preparation if they had experienced a prior colonoscopy. Repeat blood tests, including electrolyte profile, serum osmolality, blood urea nitrogen, and serum creatinine were performed after 4 days of PEG therapy in all 46 patients, before starting intravenous fluids on the day of colonoscopy. Four patients did not have osmolality tests.

Adequacy of colon preparation was assessed and agreed upon by two of the three physicians. Note was made regarding the level of endoscope insertion and any unusual colonic mucosal changes. Right and left colons were assessed separately as follows: excellent (no stool or small amount of thin fecal fluid seen), good (liquid fecal matter that can be removed by suction easily), fair (thick stools that requires suction with water lavage) or poor (solid stools present, poor visualization of the mucosa). The excellent or good colon preparation implied satisfactory visualization of the entire colonic mucosa without a need for extra effort or time to clean the colon during endoscopy.

Analysis

The stool frequency and consistency prior to PEG were compared to values on each day of PEG administration by using a paired, two-tailed Student's t test. Numerical values were expressed as mean ±SEM. Laboratory values prior to PEG therapy and after 4 days of PEG therapy were compared using a paired, two-tailed Student's t test. Categorical data were analyzed using Fisher's exact test. A P value of less than 0.05 was regarded as significant.

Results

Forty-six children (24 male, 22 female) completed the study. The mean age was 11.2 years (range 2.8 to 17.8) and the mean weight was 44 kg (range 12.1 to 87.8). The average dose of PEG solution per day was 870 ml (range 240 to 1440). Children used 2 different beverages on average (range 1 to 6) to prepare the PEG solution. The beverages used were fruit juices (30), water (12), sports drinks (11), Kool-Aid® (Kraft Foods Inc.) (11), carbonated soft drinks (10), lemonade (9), Crystal Light® (Kraft Foods Inc.) (8), and others.

Efficacy

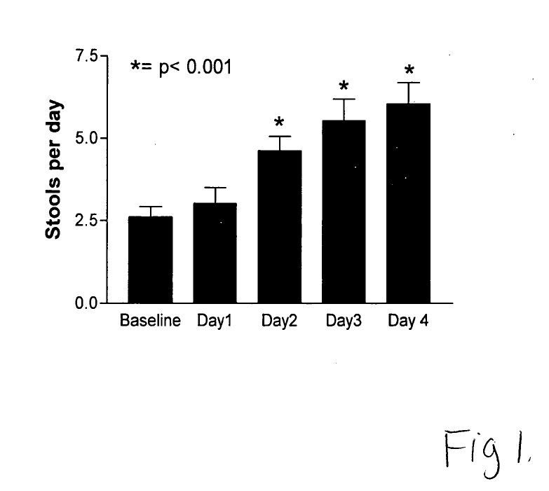

A diary of bowel movements was available for all children except one patient. FIG. 1 shows the increase in stool frequency during the PEG bowel regimen in 45 children compared to baseline. The stool consistency also increased from baseline of 3.0±0.1 to 3.3±0.2 on day 1, 3.9±0.1* on day 2, 4.3±0.1* on day 3, and 4.8±0.1* on day 4 (*=P<0.001 for the difference compared to baseline). All patients had watery stools on day 4 of the bowel preparation. During the colonoscopy, the cecum was reached in all 46 patients and the terminal ileum was reached in 30 patients. The assessments of the quality of colon preparation of colonoscopy by endoscopists are shown in Table 1. Excellent or good colon preparations (those allowing satisfactory and complete visualization of the mucosa) were seen in 91% and 95% of patients in the right and left colons, respectively. The first 25 patients received saline enemas prior to colonoscopy, however, the next 21 patients did not receive any enemas. Table 2 shows lack of effect of saline enemas (n=25 for saline enemas, n=21 for controls) with the PEG bowel regimen on endoscopic assessment of the right and left colon preparations at colonoscopy.

TABLE 1

| | Excellent | Good | Fair | Poor |
|---|---|---|---|---|
| Left Colon Preparation | 31 (67%) | 13 (28%) | 2 (4%) | 0 |
| Right Colon Preparation | 24 (52%) | 18 (39%) | 4 (9%) | 0 |
| Compliance | 41 (89%) | 4 (9%) | 1 (2%) | 0 |
| Tolerance | 39 (85%) | 6 (13%) | 1 (2%) | 0 |

TABLE 2

| | Use of enemas | No enemas | |
|---|---|---|---|
| | N = 25 | N = 21 | P value⁺ |
| Right Colon Preparation | 23 (92%) | 19 (90%) | NS |
| (Excellent or Good) | | | |
| Left Colon Preparation | 24 (96%) | 20 (95 %) | NS |
| (Excellent or Good) | | | |

⁺P value for difference not significant (NS).

Safety Profile

Thirty-two of 46 patients reported no adverse effects during 4 days of PEG. Fourteen patients reported adverse effects in the form of nausea (6), abdominal pain (6), and occasional vomiting (5) during the PEG bowel regimen. Three patients reported both nausea and abdominal pain. Out of these 14 patients, 4 patients had abdominal pain and 1 patient had nausea along with abdominal pain before starting the bowel preparation. Except for one patient with moderate abdominal pain, all adverse effects experienced during bowel preparation were rated as mild by the patients. None of the patients in the study stopped PEG because of adverse effects. At endoscopy, no unusual colonic mucosal changes were observed as have been reported with sodium phosphate bowel preparations (Zwas et al., 1996).

Physical examination prior to colonoscopy did not reveal signs of dehydration or any new clinical abnormalities in any patient. Twenty-nine patients had blood tests performed both before and after PEG therapy (Table 3). There was a small, but statistically significant decrease noted in serum potassium, carbon dioxide, and blood urea nitrogen values after the PEG bowel preparation in these 29 patients. All values, however, remained within the overall normal laboratory range. Seventeen patients had laboratory evaluation only after the PEG bowel regimen and all these blood test values were also within normal limits.

TABLE 3

| | Normal | Pre-PEG | Post-PEG | |
|---|---|---|---|---|
| Tests | Range | (n = 29) | (n = 29) | P value |
| Sodium | 135-145 | 139.7 ± 0.3 | 140.2 ± 0.5 | 0.30 |
| (mmol/L) | | | | |
| Potassium | 3.5-5.0 | 4.2 ± 0.1 | 3.9 ± 0.1 | <0.01* |
| (mmol/L) | | | | |
| Chloride | 98-106 | 103.0 ± 0.4 | 103.7 ± 0.4 | 0.21 |
| (mmol/L) | | | | |
| Carbon Dioxide | 20-28 | 24.0 ± 0.3 | 21.6 ± 0.4 | <0.001* |
| (mmol/L) | | | | |
| Urea Nitrogen | 5-18 | 11.3 ± 0.7 | 9.2 ± 0.5 | 0.01* |
| (mg/dl) | | | | |
| Creatinine | 0.4-1.0 | 0.64 ± 0.03 | 0.63 ± 0.03 | 0.85 |
| (mg/dl) | | | | |
| Osmolality | 275-295 | 285.4 ± 1.0 | 285.2 ± 1.1 | 0.92 |
| (mmol/K) | | | | |

P value for difference.
*= significant difference between pre-PEG and post-PEG values.

Patient Acceptance

The compliance and tolerance of PEG therapy reported by patients are shown in Table 1. Only one patient was unable to take the entire prescribed amount and took 67% of the prescribed PEG solution. The palatability of PEG solution was rated as either good (40) or okay (6) by all patients. Patients reported the PEG bowel preparation to be easy (35), or okay (10). One child rated it as unpleasant and none found it to be difficult. All patients except one stated willingness to retake the PEG regimen for bowel preparation for future colonoscopy if necessary. Eleven patients had colonoscopies in the past and had received previous bowel preparations with clear diet and multiple laxatives (6), PEG-ELS (4) and sodium phosphate solution (1). All of them preferred the PEG regimen to their previous bowel preparations.

Discussion

This study shows that electrolyte-free PEG can be used as an effective, safe, and palatable bowel preparation in children. Similar to previous reports (Pashankar et al., 2001; Loening-Baucke et al., 2002; Gremse et al., 2002), PEG administration led to an increase in stool frequency and a liquid stool consistency in all children. This laxative effect was significant on the second day of therapy, and by the fourth day all patients had frequent watery stools. Colon preparations were rated as excellent or good in 91% of patients in the right colon and 95% of patients in the left colon, allowing complete and satisfactory visualization of the entire colonic mucosa during endoscopy. Although normal saline enemas were initially used as a routine in the first 25 patients, it was later recognized that enemas were not necessary as colonic preparations were still satisfactory in the subsequent 21 patients without enemas. Therefore, PEG therapy for 4 days is an effective bowel preparation without the need of any enemas.

There were no major clinical adverse effects seen with the PEG bowel preparation. The adverse effects were generally mild, and no patient stopped PEG due to adverse effects. This is in agreement with previous studies showing no major adverse effects with PEG therapy in children (Pashankar et al., 2001; Loening-Baucke, 2002; Pashanker et al., 2003). Moreover, PEG did not cause any unusual colonic mucosal changes, as have been reported with sodium phosphate bowel preparations (Zwas et al., 1996).

In this study, there was no change in serum sodium, chloride, creatinine or osmolality with PEG therapy. The observed minor changes in serum potassium, carbon dioxide and urea nitrogen were clinically insignificant. Previous studies in children have reported no change in the electrolyte profile with PEG therapy (Pashankar et al., 2003; Youssef et al., 2002). In a recent study, PEG at a dose of 1.5 gm/kg/day was used for fecal disimpaction for 3 days in children (Youssef et al., 2002). There were no significant changes in the biochemical profile including electrolytes when measured 2 days after the last dose of PEG (Youssef et al., 2002). Similarly, long-term PEG therapy at a mean dose of 0.8 g/kg/day was not associated with abnormalities in the electrolyte profile, osmolality, or renal function tests in children (Pashankar et al., 2003). Studies in human volunteers indicate that systemic absorption of PEG is minimal (estimated at 0.09%) and renal excretion is high (Brady et al., 1986; DiAro et al., 1980). Therefore, systemic toxicity of PEG is unlikely and PEG therapy appears to be safe for use as bowel preparation.

The compliance with PEG therapy was excellent in 89% of children in the present study. This high compliance rate is due to the fact that PEG is virtually tasteless and children mixed it in beverages of their choice. Therefore, even though patients had to consume a relatively large volume of fluids, they tolerated the preparation quite well and none rated PEG preparation as ‘difficult’. These results are similar to previous studies showing excellent compliance and patient acceptance of long-term PEG therapy in children with constipation and encopresis (Loening-Baucke, 2002). Almost all patients stated their willingness to use PEG therapy as a bowel preparation for future colonoscopy.

Several bowel preparations have been used in children for colonoscopy, with variable limitations due to tolerance, acceptance, reliable cleansing, and adverse effects. One study reported use of 22 different bowel preparations in children, indicating lack of a safe and effective bowel preparation acceptable to children (Barrish et al., 1993). PEG-ELS is one of the standard bowel preparations used in children and adults. It usually gives excellent colonic cleansing when taken in full amount (4 liters in adults). The bowel preparation with electrolyte-free PEG therapy that was used is much different from the standard PEG-ELS regimen. PEG-ELS contains PEG 3350 and electrolytes, and is usually given at the rate of 20 to 40 ml/kg/hour for 4 hours (Sondeimer et al., 1991; Dahshan et al., 1999). PEG-ELS is not well accepted or tolerated by children because of its unpleasant salty taste and the need to consume large volume in a very short time. As a result, nausea and vomiting are very common, and nasogastric tube administration is often necessary (Sondeimer et al., 1991). In comparison, the present bowel preparation involves drinking a moderate amount of electrolyte-free PEG in a beverage of the patient's choice over 4 days. The volume consumed is about 21 ml/kg per day for 4 days. So, compared to standard PEG-ELS regimen, PEG therapy used in the present study is a much ‘gentler’ and more palatable bowel preparation.

Another bowel preparation commonly used in children involves taking a prolonged clear liquid diet and multiple doses of laxatives. Strict compliance with a clear liquid diet for 2 to 3 days is difficult for children and most laxatives have an unpleasant taste. These factors may lead to poor compliance and inadequate colonic cleansing (Dahshan et al., 1999). Sodium phosphate bowel preparation has the advantages of smaller volume and satisfactory colonic cleansing (Gremse et al., 1996; Silva et al., 1997). However, it has problems of unpleasant taste and the risk of significant hyperphosphatemia and hypocalcemia in children (Gremse et al., 1996; Silva et al., 1997; Shaoul et al., 2001). A fatality has also been reported in an adult patient taking sodium phosphate bowel preparation due to severe hyperphosphatemia (Ullah et al., 2002). Besides, sodium phosphate can induce confusing colonic mucosal changes mimicking inflammatory bowel disease in up to 24% of adult patients (Zwas et al., 1996).

As compared to other bowel preparations, the PEG regimen described herein appears to be a safe and effective bowel preparation. It has the disadvantage of requiring 4 days of bowel preparation, longer than any of the previously reported bowel preparations. However, as it is mixed in a beverage of patient's choice, patient compliance and acceptance are very satisfactory. All children in the present study preferred PEG to the other bowel preparations they had used for colonoscopy in the past. Therefore, polyethylene glycol 3350 without electrolytes should be considered an attractive option for bowel preparation for colonoscopy in children.

References

Barrish et al., Gastroenetrol. Nurs., 16:106 (1993).

Brady et al., Gastroenterology, 90:1914 (1986).

Dahshan et al., Am. J. Gastroenterol., 94:3497 (1999).

DiPiro et al., Clin. Pharm., 5:153 (1986).

Gremse et al., Clin. Pediatr., 41:225 (2002).

Gremse et al., J. Pediatr. Gastroenterol. Nutr., 23:586 (1996).

Loening-Baucke, J. Pediatr. Gastroenterol. Nutr., 34:372 (2002).

Pashankar et al., Arch. Pediatr. Adolesc. Med., 157:661 (2003).

Pashankar et al., Clin. Pediatr., 42:815 (2003).

Pashankar et al., J. Pediatr., 139:428 (2001).

Pinefield et al., Arch. Dis. Child., 80:181 (1999).

Shaoul et al., Gastrointest. Endosc., 53:650 (2001).

Silva et al., J. Pediatr. Gastroenterol. Nutr., 24:33 (1997).

Sondeimer et al., J. Pediatr., 119:148 (1991).

Ullah et al., J. Clin. Gastroenterol., 34:457 (2002).

Youssef et al., J. Pediatr., 141:410 (2002).

Zwas et al., Gastrointest. Endosc. 43:463 (1996).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to induce colon cleansing comprising: prior to endoscopy administering to a patient in need thereof a composition comprising an effective amount of electrolyte-free polyethylene glycol (PEG).

2. The method of claim 1 wherein the amount reduces or eliminates stool in the colon.

3. The method of claim 1 wherein the amount results in no stool or a small amount of thin fecal fluid in the colon during endoscopy.

4. The method of claim 1 wherein the amount provides for complete visualization of the colonic mucosa.

5. The method of claim 1 wherein the patient is a child.

6. The method of claim 1 wherein the amount is administered over two or more days.

7. The method of claim 1 wherein the dosage per day is about 0.5 to 2.5 g/kg.

8. The method of claim 1 wherein the dosage per day is about 1.0 to 2.0 g/kg.

9. The method of claim 1 wherein the PEG is dispersed in an aqueous medium.

10. The method of claim 9 wherein the dosage of PEG per day is dispersed in a volume of about 100 mL to about 2000 mL.

11. The method of claim 1 wherein the PEG is PEG 3350.

12. The method of claim 1 wherein the dosage is 5 to 200 g/day.

13. The method of claim 1 wherein the dosage is 50 to 150 g/day.

14. The method of claim 1 wherein the patient is not pretreated with an enema.

15. The method of claim 1 wherein the electrolytes of the patient are substantially unaffected by the administration.

16. The method of claim 1 wherein the composition is administered orally.

17. The method of claim 1 wherein the PEG has an average molecular weight greater than about 1000.

18. The method of claim 1 wherein the PEG has an average molecular weight from about 3000 to about 8000.

19. The method of claim 1 wherein the patient has diarrhea, abdominal pain and/or blood in stool.
